(19) United States
(12) Patent Application Publication
(10) Pub. No.: US 2026/0248822 A1
(43) Pub. Date: Aug. 27, 2026

(54) METHOD AND COMPOSITION FOR THE TREATMENT OF CANCER

(71) Applicant: IMPACT HEALTH GLOBAL LTD., Ashdod (IL)

(72) Inventor: Khaled Khalil, Ashdod (IL)

(21) Appl. No.: 19/160,371

(22) PCT Filed: Feb. 26, 2024
(86) PCT No.: PCT/IL2024/050214

§ 371 Date: Aug. 27, 2025

(30) Foreign Application Priority Data
Feb. 28, 2023 (IL) 301010

(51) Int. Cl. A61K 31/00; A61K 31/352; A61K 31/56; A61K 31/7028; A61P 35/00

(52) U.S. Cl. CPC A61K 31/658; A61K 31/352; A61K 31/56; A61K 31/7028; A61P 35/00

(57) ABSTRACT

The present invention relates to a method for treatment of cancer, e.g., breast cancer, by administration of an active agent combination comprising sinigrin, vitexin, taraxasterol, and a cannabinoid such as CBD, as well as to a kit for carrying out said method, and a composition comprising said active agent combination.

### TECHNICAL FIELD

The present invention relates to a method for treatment of cancer, more particularly breast cancer, by administration of an active agent combination, as well as to a kit for carrying out said method, and a composition comprising said active agent combination.

### BACKGROUND ART

Breast cancer, which may start in either one or both breasts, is considered the most common cancer in women in the United States, except for skin cancers, and it is about 30% of all new female cancers each year. Overall, the average risk of a woman in the United States developing breast cancer sometime in her life is about 13%. The tumor growth and metastatic spread of 4T1 cells in BALB/c mice very closely mimic human breast cancer. This tumor-derived cell line is an animal model for stage IV human breast cancer.

Sinigrin (((Z)-N-[1-(β-D-glucopyranosylsulfanyl)but-3-en-1-ylidene] hydroxylamine-O-sulfonic acid), also known as allyl-glucosinolate or 2-propenyl-glucosinolate, is a natural aliphatic glucosinolate found in cruciferous vegetables such as broccoli, and has been shown to have anti-cancer properties. As disclosed in Park et al., 2011, sinigrin induces apoptosis in human lung cancer cells.

Vitexin (8-(β-D-glucopyranosyl)-4′,5,7-trihydroxyflavone) is an apigenin flavone glucoside, a chemical compound found in plants such as chamomile, and has been shown to have anti-cancer properties. As disclosed in Jang et al., 2012, vitexin induces apoptosis in human lung cancer cells and suppresses the growth of tumors in mice.

Taraxasterol ((3S,4aR,6aR,6bR,8aR,12S,12aR,12bR,14aR,14bR)-4,4,6a,6b,8a,12, 14b-heptamethyl-11-methylidenedocosahydropicen-3-ol; 18α,19α-Urs-20(30)-en-3β-ol), also known as anthesterin, is a triterpene derived from the mevalonate pathway and is found in plants such as dandelions, and has been shown to have anti-cancer properties. According to Kim et al., 2016, taraxasterol induces apoptosis and suppresses tumor growth in human colon cancer cells.

WO 2020075170 discloses a combination comprising a Taraxacum plant extract, which includes taraxasterol, and a cannabinoid such as CBD, or an enantiomer, diastereomer, or racemate thereof, for use in the treatment of cancer.

### SUMMARY OF INVENTION

It has been found, in accordance with the present invention, that a combination of sinigrin, vitexin and taraxasterol has a synergistic effect in killing breast cancer cells in vitro, compared to each one of said active agents alone; and that such a combination, when administered together with CBD, is highly effective and significantly more effective than said combination when administered without CBD, in reducing both the weight and the volume of breast tumor, and may thus be beneficial in treatment of cancer, e.g. breast cancer.

The present invention thus generally refers to at least two of sinigrin, vitexin and taraxasterol, i.e., sinigrin and vitexin; sinigrin and taraxasterol; vitexin and taraxasterol; or sinigrin, vitexin and taraxasterol, and a cannabinoid such as CBD or an enantiomer, diastereomer, or racemate thereof, for use as a combination in the treatment of cancer.

In one specific aspect, the present invention relates to a method for treatment of cancer in a subject in need thereof, said method comprising administering to said subject a therapeutically effective amount of a combination of sinigrin, vitexin, taraxasterol, and a cannabinoid. In particular embodiments, the cannabinoid administered according to said method is CBD, or an enantiomer, diastereomer, or racemate thereof, preferably CBD.

The cancer treated by the method disclosed may be any solid tumor such as both melanoma- and non-melanoma-skin cancer, lung cancer, prostate cancer, breast cancer, pancreatic cancer such as adenocarcinoma of the pancreas, gastric cancer, colorectal cancer such as colorectal carcinoma, kidney (renal) cancer such as renal carcinoma, bladder cancer, gallbladder cancer, liver cancer, brain cancer such as glioblastoma, multiple myeloma, leukemia, both Hodgkin's-and non-Hodgkin's-lymphoma, and head and neck tumors, as well as metastases thereof. In particular embodiments, said cancer is breast cancer, e.g., luminal A breast cancer, luminal B breast cancer, human epidermal growth factor receptor 2 (HER2)-positive breast cancer, and triple-negative breast cancer.

In another specific aspect, the present invention relates to sinigrin, vitexin, taraxasterol, and a cannabinoid, for use as a combination in the treatment of cancer. In particular embodiments, said cannabinoid is CBD, or an enantiomer, diastereomer, or racemate thereof, preferably CBD.

In a further specific aspect, disclosed herein is a kit comprising at least two compositions, each comprising one or more of sinigrin, vitexin, taraxasterol, and a cannabinoid, wherein each one of said compositions is independently formulated for oral or parenteral administration, but excluding a kit comprising a composition comprising said cannabinoid as a sole active agent, and a composition comprising said taraxasterol as a sole active agent. In certain embodiments, said kit comprises a first composition comprising said cannabinoid formulated, e.g., for oral administration; and a second composition comprising said sinigrin, said vitexin, and said taraxasterol, formulated for either oral or parenteral administration. Particular such kits are those wherein said cannabinoid is CBD, or an enantiomer, diastereomer, or racemate thereof, preferably CBD.

In yet a further specific aspect, the present invention provides a composition comprising sinigrin, vitexin, taraxasterol, and a cannabinoid. In particular embodiments, said cannabinoid is CBD, or an enantiomer, diastereomer, or racemate thereof, preferably CBD. Such a composition is useful in treatment of cancer, more specifically a solid tumor such as those listed above, or a metastasis thereof.

### BRIEF DESCRIPTION OF DRAWINGS

FIGS. 1A-1B show MTT assay in vitro. 1A shows that treatment with each one of taraxasterol, vitexin and sinigrin, at a concentration of 2 mM, reduced cancer cell viability compared to the control (taraxasterol 29%, vitexin 20%, sinigrin 23%), whereas the mixture of the three components reduced cell viability by 48% in 48 hours. 1B shows the 4T1 viability for the various treatments at 2 mM concentrations, 48 hours post administration.

FIG. 2 shows representative pictures of the tumors at Day 19 of the in vivo experiment described in Example 2, in both the vehicle group and the group treated with the taraxasterol, vitexin and sinigrin mixture and CBD (Mix+CBD), vs. a healthy mouse.

FIGS. 3A-3D show average tumor volume during the in vivo experiment described in Example 2 (left panel), and tumor volume at Day 27 (right panel) (3A); average mouse weight during said experiment (3B); average tumor weight at Day 28 of said experiment (3C); and pictures of representative tumors extracted from the three groups after termination of said experiment (3D).

### DETAILED DESCRIPTION

In one specific aspect, disclosed herein is a method for treatment of cancer in a subject in need thereof, said method comprising administering to said subject a therapeutically effective amount of a combination of sinigrin, vitexin, taraxasterol, and a cannabinoid.

The active agents/components other than said cannabinoid, administered according to the method of the present invention, i.e., sinigrin, vitexin and taraxasterol, are also together referred to herein as “non-cannabinoid components”, and the overall amount of said active agents administered to said subject is also referred to herein as “the overall amount of the non-cannabinoid components”.

The term “cannabinoid” as used herein refers to a chemical compound acting on a cannabinoid receptor, i.e., a cannabinoid type 1 (CB1) and/or cannabinoid type 2 (CB2) receptor. Ligands for these receptor proteins include the endocannabinoids produced naturally in the body; the phytocannabinoids found in Cannabis sativa, Cannabis indica, Cannabis ruderalis, and some other plants; and synthetic cannabinoids.

The cannabinoid used according to the present invention, in combination with sinigrin, vitexin and taraxasterol, may be derived from a Cannabis extract using any suitable extraction and purification procedures known in the art. Alternatively, said cannabinoid may be synthesized following any one of the procedures disclosed in the literature.

In certain embodiments, the cannabinoid administered according to the method of the present invention, in combination with sinigrin, vitexin and taraxasterol, is selected from cannabidiol (CBD), cannabidiolic acid (CBDA), cannabidivarin (CBDV), cannabidivarinic acid (CBDVA), cannabidiol monomethyl ether (CBDM), cannabidiol-C4 (CBD-C4), cannabidiorcol (CBD-C1), Δ⁹-tetrahydrocannabinol (Δ⁹-THC), Δ⁹-tetrahydrocannabinolic acid (Δ⁹-THCA), Δ⁹-tetrahydrocannabivarin (Δ⁹-THCV), Δ⁹-THCVA, Δ⁸-THC, Δ⁸-THCA, Δ⁸-THCV, Δ⁸-THCVA, iso-tetrahydrocannabinol-type (iso-THC), cannabinol (CBN), cannabinolic acid (CBNA), cannabinol-C4 (CBN-C4), cannabinol-C2 (CBN-C2), cannabiorcol (CBN-C1), cannabinol methyl ether (CBNM), cannabinodiol (CBND), cannabigerol (CBG), cannabigerovarin (CBGV), cannabigerolic acid (CBGA), cannabigerovarinic acid (CBGVA), cannabigerol monomethyl ether (CBGM), cannabigerolic acid monomethyl ether (CBGAM), cannabichromene (CBC), cannabichromenic acid (CBCA), cannabichromevarin (CBCV), cannabichromevarinic acid (CBCVA), cannabichromanon (CBCN), cannabicyclol (CBL), cannabicyclolic acid (CBLA), cannabicyclovarin (CBLV), cannabivarin (CBV), cannabivarinic acid (CBVA), cannabielsoin (CBE), cannabielsoic acid A (CBEA-A), cannabielsoic acid B (CBEA-B), cannabitriol (CBT), cannabitriolvarin (CBTV), ethoxy-cannabitiolvarin (CBTVE), cannabifuran (CBF), dehydrocannabifuran (DCBF), cannabiripsol (CBR), an enantiomer, diastereomer, racemate, or pharmaceutically acceptable salt thereof, and a combination thereof.

For the sake of simplicity and if not otherwise explicitly specified, all references made throughout this specification to a particular cannabinoid of those listed hereinabove, e.g., CBD, further refer to an enantiomer, diastereomer, mixture, e.g., racemate, or a pharmaceutically acceptable salt of said cannabinoid.

In particular embodiments, the cannabinoid administered according to the method of the present invention, in combination with sinigrin, vitexin and taraxasterol, is CBD (2-[(1R,6R)-6-isopropeny 1-3-methylcyclohex-2-en-1-yl]-5-pentylbenzene-1,3-diol), or an enantiomer, diastereomer, or racemate thereof, but it is preferably 2-[(1R,6R)-6-isopropenyl-3-methylcyclohex-2-en-1-yl]-5-pentylbenzene-1,3-diol. CBD has two stereogenic centers, i.e., at positions 3 and 4 of the cyclohexenyl ring, and may accordingly exist as an enantiomer, i.e., an optical isomer (R or S, which may have an optical purity of 90%, 95%, 99% or more), racemate, i.e., an optically inactive mixture having equal amounts of R and S enantiomers, a diastereoisomer, or a mixture thereof. The present invention encompasses the use of all such enantiomers, isomers and mixtures thereof.

CBD may be synthesized following any one of the procedures known in the art, e.g., by acid condensation of p-mentha-2,8-dien-1-ol with olivetol. Optically active forms of CBD may be prepared using any one of the methods disclosed in the art, e.g., by resolution of the racemic form by recrystallization techniques; chiral synthesis; extraction with chiral solvents; or chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

In certain embodiments, the ratio between the amounts of sinigrin, vitexin and taraxasterol administered according to the method disclosed is in a range of 0.5-2.0: 0.5-2.0: 0.5-2.0, e.g., in a range of 0.6-1.6: 0.6-1.6: 0.6-1.6, in a range of 0.7-1.4: 0.7-1.4: 0.7-1.4, or in a range of 0.8-1.2: 0.8-1.2: 0.8-1.2, respectively, by weight. In particular such embodiments, the ratio between the amounts of sinigrin, vitexin and taraxasterol administered is in a range of 0.9-1.1: 0.9-1.1: 0.9-1.1, preferably about 1:1:1, respectively, by weight.

In certain embodiments, the ratio between the overall amount of sinigrin, vitexin and taraxasterol, i.e., the overall amount of the non-cannabinoid components, and the amount of said cannabinoid, administered according to the method disclosed, is in a range of 20:1 to 50:1, e.g., in a range of 25:1 to 35:1, or in a range of 28:1 to 32:1, respectively, by weight. In particular such embodiments, the ratio between the overall amount of the non-cannabinoid components and the amount of said cannabinoid administered is in a range of 29:1 to 31:1, but preferably about 30:1, respectively, by weight.

In certain embodiments, the cannabinoid administered according to the method of the present invention is CBD; the ratio between the amounts of sinigrin, vitexin and taraxasterol administered according to said method is in a range of 0.5-2.0: 0.5-2.0: 0.5-2.0, e.g., in a range of 0.6-1.6: 0.6-1.6: 0.6-1.6, in a range of 0.7-1.4: 0.7-1.4: 0.7-1.4, or in a range of 0.8-1.2: 0.8-1.2: 0.8-1.2, respectively, by weight; and the ratio between the overall amount of the non-cannabinoid components and the amount of said CBD administered according to said method is in a range of 20:1 to 50:1, e.g., in a range of 25:1 to 35:1, or in a range of 28:1 to 32:1, respectively, by weight. In particular such embodiments, the ratio between the amounts of sinigrin, vitexin and taraxasterol administered is in a range of 0.9-1.1: 0.9-1.1: 0.9-1.1, preferably about 1:1:1, respectively, by weight; and the ratio between the overall amount of the non-cannabinoid components and the amount of said CBD administered is in a range of 29:1 to 31:1, but preferably about 30:1, respectively, by weight. In certain such embodiments, the cannabinoid administered is 2-[(1R,6R)-6-isopropeny1-3-methylcyclohex-2-en-1-yl]-5-pentylbenzene-1,3-diol.

The method of the present invention comprises administering to a subject in need thereof a cannabinoid as defined in any one of the embodiments above, more particularly CBD, or an enantiomer, diastereomer, or racemate thereof, but preferably CBD, in combination with sinigrin, vitexin and taraxasterol, wherein the weight ratio between the amounts of sinigrin, vitexin and taraxasterol administered, and the weight ratio between the overall amount of the non-cannabinoid components and the amount of said cannabinoid administered, each independently is as defined in any one of the embodiments above. According to the present invention, the cannabinoid and the non-cannabinoid components administered as a combination may be formulated as two or more separate compositions, for the same or different administration mode (e.g., wherein one composition comprises said cannabinoid, and a second composition comprises said non-cannabinoid components), which may be administered concomitantly, or sequentially at any order. Alternatively, the combination of said cannabinoid and said non-cannabinoid components may be formulated as a sole composition. It should be understood that regardless of the number of compositions administered, each one of the compositions may independently be a pharmaceutical or nutraceutical composition.

In particular embodiments, the combination of active agents administered according to the method of the present invention, i.e., said cannabinoid and said non-cannabinoid components, are formulated as two or more separate compositions, wherein each one of said compositions is formulated for oral or parenteral administration. In a particular such embodiment, said cannabinoid is formulated as a first composition formulated for oral administration, and said non-cannabinoid components are formulated as a second composition formulated for either oral or parenteral administration.

The method of the present invention is aimed at treating a cancer in a subject in need thereof.

The term “subject” as used herein refers to any mammal, e.g., a human, non-human primate, horse, ferret, dog, cat, cow, and goat. In a preferred embodiment, the term “subject” denotes a human, i.e., an individual.

The term “cancer” as used herein refers to both a solid tumor as well as a metastasis thereof and includes, e.g., both melanoma-and non-melanoma-skin cancer, lung cancer, prostate cancer, breast cancer, pancreatic cancer such as adenocarcinoma of the pancreas, gastric cancer, colorectal cancer such as colorectal carcinoma, kidney (renal) cancer such as renal carcinoma, bladder cancer, gallbladder cancer, liver cancer, brain cancer such as glioblastoma, multiple myeloma, leukemia, both Hodgkin's-and non-Hodgkin's-lymphoma, and head and neck tumors. In particular embodiments, the cancer treated by the method of the present invention is breast cancer such as, without being limited to, luminal A breast cancer, luminal B breast cancer, human epidermal growth factor receptor 2 (HER2)-positive breast cancer, and triple-negative breast cancer.

In a particular such aspect, disclosed herein is a method for treatment of breast cancer, e.g., luminal A breast cancer, luminal B breast cancer, human epidermal growth factor receptor 2 (HER2)-positive breast cancer, and triple-negative breast cancer, in a subject in need thereof, said method comprising administering to said subject a therapeutically effective amount of a combination of sinigrin, vitexin, taraxasterol, and a cannabinoid or an enantiomer, diastereomer, racemate, or pharmaceutically acceptable salt thereof, e.g., CBD, or an enantiomer, diastereomer, or racemate thereof, preferably CBD. In certain embodiments, the weight ratio between the amounts of sinigrin, vitexin and taraxasterol administered, and the weight ratio between the overall amount of the non-cannabinoid components and the amount of said cannabinoid administered, each independently is as defined in any one of the embodiments above.

The term “treatment” as used herein refers to the administering of a therapeutic amount of a drug (i.e., active agent) or drug combination, e.g., the combination of the cannabinoid and non-cannabinoid components referred to herein, which is effective to ameliorate undesired symptoms associated with the medical condition treated; prevent the manifestation of such symptoms before they occur; slow down the progression of said medical condition; slow down the deterioration of symptoms; enhance the onset of remission period; slow down the irreversible damage caused in the progressive chronic stage of said medical condition; delay the onset of said progressive stage; lessen the severity or cure said medical condition; improve survival rate or more rapid recovery; and/or prevent said medical condition form occurring.

The term “therapeutically effective amount” as used herein means an amount or dose of an active agent or active agent combination, e.g., the combination of the cannabinoid and non-cannabinoid components referred to herein, that is useful to treat, attenuate, prevent, or delay the onset of at least one pathological phenotype manifested by cancer. The amount must be effective to achieve the desired therapeutic effect as described above, depending, inter alia, on the type and severity of the condition to be treated and the treatment regime. The therapeutically effective amount is typically determined in appropriately designed clinical trials (dose range studies) and the person versed in the art will know how to properly conduct such trials to determine said amount.

In certain embodiments, the daily dose for human of each one of the non-cannabinoid components, i.e., sinigrin, vitexin and taraxasterol, is from about 50 mg to about 150 mg, e.g., from about 80 mg to about 120 mg, from about 90 mg to about 110 mg, or about 100 mg; and the daily dose of the cannabinoid administered, e.g., CBD, is from about 5 mg to about 15 mg, e.g., from about 7 mg to about 13 mg, from about 8 mg to about 12 mg, from about 9 mg to about 11 mg, or about 10 mg.

As shown herein, administration of a combination of a cannabinoid, more specifically CBD, and the non-cannabinoid components referred to herein, per se, is highly beneficial in treatment of cancer such as breast cancer. Yet, it should be noted that such a treatment may also be provided as an adjunctive therapy to a conventional therapy aimed at treating cancer, e.g., a breast cancer. The term “conventional therapy” as used herein refers to any conventional treatment of cancer, e.g., breast cancer, by the use of a chemotherapy, hormonotherapy, immunotherapy, tyrosine kinase inhibitor-based therapy, antiangiogenic based-therapy, antibody-based therapy, or a combination thereof. The term “adjunctive therapy” as used herein refers to a therapy given to a subject having a cancer, e.g., a breast cancer, and undergoing a conventional therapy for treating said cancer, in addition and in parallel to said conventional therapy, so as to increase or maximize the efficacy of said conventional therapy. Such an adjunctive therapy may have even a greater synergistic effect increasing or maximizing the efficacy of said conventional therapy.

Thus, in certain embodiments, disclosed herein is a method for treatment of cancer, e.g., a breast cancer, in a subject in need thereof, according to any one of the embodiments above, wherein said subject is undergoing a conventional therapy such as a chemotherapy, hormonotherapy, immunotherapy, tyrosine kinase inhibitor-based therapy, antiangiogenic based-therapy, antibody-based therapy, or a combination thereof, for treating said cancer, and said combination of sinigrin, vitexin, taraxasterol, and a cannabinoid is administered to said subject as an adjunctive therapy.

In another specific aspect, disclosed herein is sinigrin, vitexin, taraxasterol, and a cannabinoid, for use as a combination in the treatment of cancer. In particular embodiments, said cannabinoid is CBD, or an enantiomer, diastereomer, or racemate thereof, but preferably CBD. In certain embodiments, the weight ratio between the amounts of sinigrin, vitexin and taraxasterol administered, and the weight ratio between the overall amount of the non-cannabinoid components and the amount of said cannabinoid administered, each independently is as defined in any one of the embodiments above.

In a further specific aspect, disclosed herein is a kit comprising at least two compositions, each comprising one or more of sinigrin, vitexin, taraxasterol, and a cannabinoid, wherein each one of said compositions is independently formulated for oral or parenteral administration, but excluding a kit comprising a composition comprising said cannabinoid as a sole active agent, and a composition comprising said taraxasterol as a sole active agent.

In certain embodiments, the kit of the present invention comprises a first composition comprising said cannabinoid formulated, e.g., for oral administration; and two additional compositions each independently formulated for either oral or parenteral administration, wherein one of said additional compositions comprises one of said sinigrin, said vitexin, and said taraxasterol, and the other one of said additional compositions comprises the others of said sinigrin, said vitexin, and said taraxasterol.

In other embodiments, the kit of the present invention comprises a first composition comprising said cannabinoid formulated, e.g., for oral administration; and a second composition comprising said sinigrin, said vitexin, and said taraxasterol, formulated for either oral or parenteral administration.

In particular embodiments, the compositions comprised within the kit of the present invention, according to any one of the embodiments above, contain said cannabinoid and said non-cannabinoid components in amounts such that the ratio between the amounts of sinigrin, vitexin and taraxasterol is in a range of 0.5-2.0: 0.5-2.0: 0.5-2.0, e.g., in a range of 0.6-1.6: 0.6-1.6: 0.6-1.6, in a range of 0.7-1.4: 0.7-1.4: 0.7-1.4, or in a range of 0.8-1.2: 0.8-1.2: 0.8-1.2, respectively, by weight; and the ratio between the overall amount of the non-cannabinoid components and the amount of said cannabinoid is in a range of 20:1 to 50:1, e.g., in a range of 25:1 to 35:1, or in a range of 28:1 to 32:1, respectively, by weight. In particular such embodiments, said compositions contain said cannabinoid and said non-cannabinoid components in amounts such that the ratio between the amounts of sinigrin, vitexin and taraxasterol is in a range of 0.9-1.1: 0.9-1.1: 0.9-1.1, preferably about 1:1:1, respectively, by weight; and the ratio between the overall amount of the non-cannabinoid components and the amount of said cannabinoid is in a range of 29:1 to 31:1, but preferably about 30:1, respectively, by weight.

In certain embodiments, the cannabinoid comprised within the kit of the present invention, according to any one of the embodiments above, is CBD, or an enantiomer, diastereomer, or racemate thereof, preferably CBD.

In yet a further specific aspect, disclosed herein is a composition comprising sinigrin, vitexin, taraxasterol, and a cannabinoid. In particular embodiments, said cannabinoid is CBD, or an enantiomer, diastereomer, or racemate thereof, but preferably CBD.

In certain embodiments, the ratio between the amounts of sinigrin, vitexin and taraxasterol comprised within the composition disclosed is in a range of 0.5-2.0: 0.5-2.0: 0.5-2.0, e.g., in a range of 0.6-1.6: 0.6-1.6: 0.6-1.6, in a range of 0.7-1.4: 0.7-1.4: 0.7-1.4, or in a range of 0.8-1.2: 0.8-1.2: 0.8-1.2, respectively, by weight. In particular such embodiments, the ratio between the amounts of sinigrin, vitexin and taraxasterol administered is in a range of 0.9-1.1: 0.9-1.1: 0.9-1.1, preferably about 1:1:1, respectively, by weight.

In certain embodiments, the ratio between the overall amount of the non-cannabinoid components and the amount of said cannabinoid, comprised within the composition disclosed, is in a range of 20:1 to 50:1, e.g., in a range of 25:1 to 35:1, or in a range of 28:1 to 32:1, respectively, by weight. In particular such embodiments, the ratio between the overall amount of the non-cannabinoid components and the amount of said cannabinoid administered is in a range of 29:1 to 31:1, but preferably about 30:1, respectively, by weight.

In certain embodiments, the cannabinoid comprised within the composition disclosed is CBD; the ratio between the amounts of sinigrin, vitexin and taraxasterol in said composition in a range of 0.5-2.0: 0.5-2.0: 0.5-2.0, e.g., in a range of 0.6-1.6: 0.6-1.6: 0.6-1.6, in a range of 0.7-1.4: 0.7-1.4: 0.7-1.4, or in a range of 0.8-1.2: 0.8-1.2: 0.8-1.2, respectively, by weight; and the ratio between the overall amount of the non-cannabinoid components and the amount of said CBD in said composition is in a range of 20:1 to 50:1, e.g., in a range of 25:1 to 35:1, or in a range of 28:1 to 32:1, respectively, by weight. In particular such embodiments, the ratio between the amounts of sinigrin, vitexin and taraxasterol in said composition is in a range of 0.9-1.1: 0.9-1.1: 0.9-1.1, preferably about 1:1:1, respectively, by weight; and the ratio between the overall amount of the non-cannabinoid components and the amount of said CBD in said composition is in a range of 29:1 to 31:1, but preferably about 30:1, respectively, by weight.

Compositions as referred to herein may be either pharmaceutical or nutraceutical compositions, and may be prepared by conventional techniques, e.g., as described in Remington: The Science and Practice of Pharmacy, 19^{th} Ed., 1995. More specifically, such compositions may be prepared, e.g., by uniformly and intimately bringing the one or more of the active agents, i.e., one or more of the cannabinoid and the non-cannabinoid components, into association with a liquid carrier, a finely divided solid carrier, or both, and then, if necessary, shaping the product into the desired formulation. The composition may be in liquid, solid or semisolid form and may further include various acceptable fillers, carriers, diluents or adjuvants, and other inert ingredients and excipients.

Compositions as referred to herein may be formulated for any suitable route of administration, including both oral and parenteral administration, but are preferably formulated for oral, sublingual, buccal, or rectal administration.

The compositions referred to herein, when formulated for oral administration, may be in any suitable form, e.g., tablets, troches, lozenges, aqueous, or oily suspensions, dispersible powders or granules, emulsions, hard or soft capsules, or syrups or elixirs. In certain embodiments, said tablets are in the form of matrix tablets in which the release of a soluble active is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other embodiments, the tablets are formulated as bi-or multi-layer tablets, made up of two or more distinct layers of granulation compressed together with the individual layers lying one on top of another, with each separate layer containing a different active agent. Bilayer tablets have the appearance of a sandwich since the edge of each layer or zone is exposed.

Compositions for oral administration might be formulated so as to inhibit the release of one or both of the active agents in the stomach, i.e., delay the release of one or both of the active agents until at least a portion of the dosage form has traversed the stomach, in order to avoid the acidity of the gastric contents from hydrolyzing the active agents. Particular such compositions are those wherein the active agent(s) is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly (methacrylicacid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

In certain embodiments, the invention provides a composition for oral administration, which is solid and may be in the form of granulate, granules, grains, beads or pellets, mixed and filled into capsules or sachets, or compressed to tablets by conventional methods. In some particular embodiments, the composition is in the form of a bi- or multilayer tablet, in which each one of the layers comprise one of the two active agents, and the layers are optionally separated by an intermediate, inactive layer, e.g., a layer comprising one or more disintegrants.

Another contemplated formulation is depot systems, based on biodegradable polymers. As the polymer degrades, the active agent(s) is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

Compositions for oral administration may be prepared according to any method known to the art and may further comprise one or more agents selected from sweetening agents, flavoring agents, coloring agents and preserving agents in order to provide pharmaceutically elegant and palatable preparations. Tablets contain the active agents in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

Useful dosage forms of the pharmaceutical compositions include orally disintegrating systems including, but not limited to, solid, semi-solid and liquid systems including disintegrating or dissolving tablets, soft or hard capsules, gels, fast dispersing dosage forms, controlled dispersing dosage forms, caplets, films, wafers, ovules, granules, buccal/mucoadhesive patches, powders, freeze dried (lyophilized) wafers, chewable tablets which disintegrate with saliva in the buccal/mouth cavity and combinations thereof. Useful films include, but are not limited to, single layer stand-alone films and dry multiple layer stand-alone films.

The composition referred to herein may comprise one or more pharmaceutically-or nutraceutically acceptable excipients. For example, a tablet may comprise at least one filler, e.g., lactose, ethylcellulose, microcrystalline cellulose, silicified microcrystalline cellulose; at least one disintegrant, e.g., cross-linked polyvinylpyrrolidinone; at least one binder, e.g., polyvinylpyridone, hydroxypropylmethyl cellulose; at least one surfactant, e.g., sodium laurylsulfate; at least one glidant, e.g., colloidal silicon dioxide; and at least one lubricant, e.g., magnesium stearate.

The composition referred to herein may be formulated for controlled release of one or more of the active agents. Such compositions may be formulated as controlled-release matrix, e.g., as controlled-release matrix tablets in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other configurations, the compositions comprise the active agent formulated for controlled release in microencapsulated dosage form, in which small droplets of the active agent are surrounded by a coating or a membrane to form particles in the range of a few micrometers to a few millimeters.

Unless otherwise indicated, all numbers expressing, e.g., weight or volume ratios of the active agents, or dosages, used in this specification, are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting Examples.

### EXAMPLES

### Materials and Methods

Singrin (≥98% purity) was purchased from Chongqing Chemdad Co., Ltd. (China). Vitexin and taraxasterol (both at ≥98% purity) were purchased from Chongqing Xingcan Pharmaceutical (Group 2, Dongfanghong village, Jiangjin District, Chongqing, China). CBD (40 mg/mL full spectrum active cannabinoids dissolved in hemp seed oil and medium chain triglycerides) was purchased from Ananda Hemp, PO Box 648, Cynthiana, KY 41031.

### Example 1. Assay Protocol-In Vitro Experiment

Mouse breast carcinoma model (4T1 cells) were cultured in the presence of taraxasterol, vitexin, sinigrin or a mixture of all these molecules, at the indicated concentrations (0.5, 1, 1.5, and 2 mM), for 48 hours. Cell viability was determined by MTT colorimetric assay, in which a yellow tetrazole is reduced to purple formazan in living cells. The resultant color was measured at 450 nm. Reduced viability of cancer cells was a marker for the efficacy of the treatment.

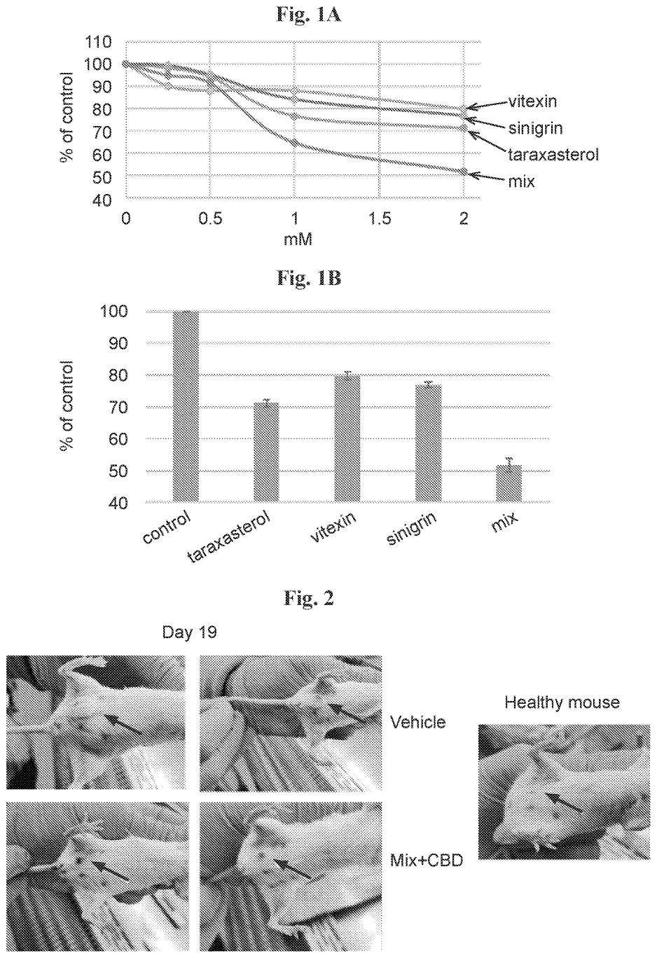

As shown in FIG. 1, treatment with each one of taraxasterol, vitexin, sinigrin, or the combination thereof reduced cancer cell viability dose dependently (compared to the control). At the highest concentration (2 mM), taraxasterol reduced cell viability by 29%, vitexin reduced cell viability by 20%, and sinigrin reduced cell viability by 23%. The combination of the three molecules reduced cell viability by 48% in 48 h treatment.

### Example 2. Assay Protocol-In Vivo Experiment

4T1 cells (1.5×105 cells in 50 μL PBS), were inoculated subcutaneously into the thigh of the mice, in the mammary gland area (n=10 in each group).

A control solution has been prepared, by mixing 250 μL PBS, 50 μL water, 50 μL DMSO, and 25 μL ethanol.

Treatment began when the size of the tumor developed has reached 0.5 cm. Specifically, the mice of the control (vehicle) group were administered with the control solution (25 μL per mouse); and the mice of the other two groups were administered orally with a mixture of 1.76 mg/kg of each one of taraxasterol, vitexin and sinigrin, either alone (Mix group) or in combination with 0.176 mg/kg CBD (Mix+CBD group) (in each case, overall dose of 25 μL per mouse), once a day, 6 days/week, starting at Day 9 until the end of the experiment (Day 26). The tumor volume and mice weight were measured twice a week. After termination of the experiment, tumor weight was measured, and tumors were fixated for future evaluation. Reduced tumor volume and tumor weight are markers for the efficacy of the treatment.

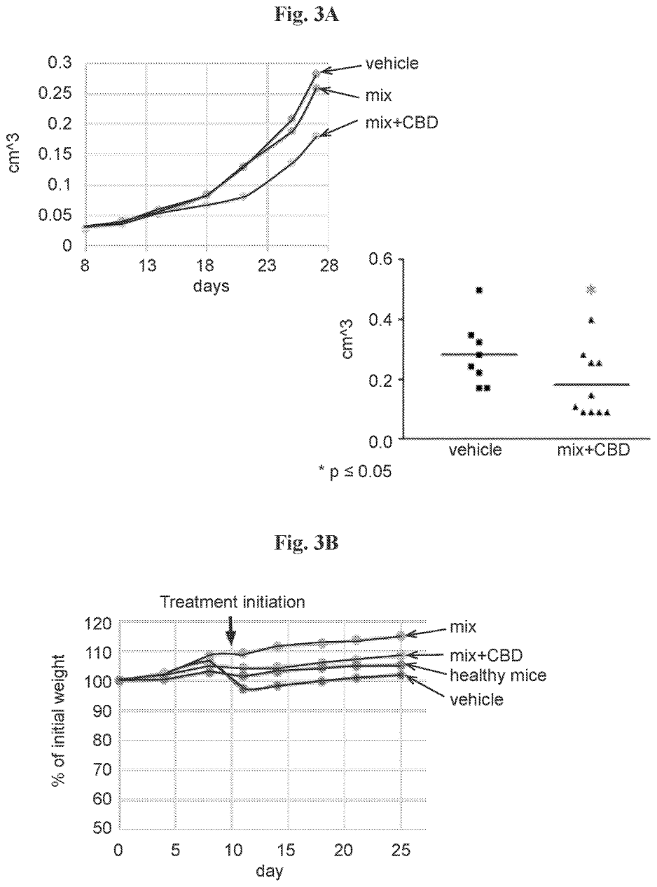

FIG. 2 shows representative pictures of the tumors at Day 19 of the experiment in both the vehicle group and the group treated with the taraxasterol, vitexin and sinigrin mixture and CBD, vs. a healthy mouse.

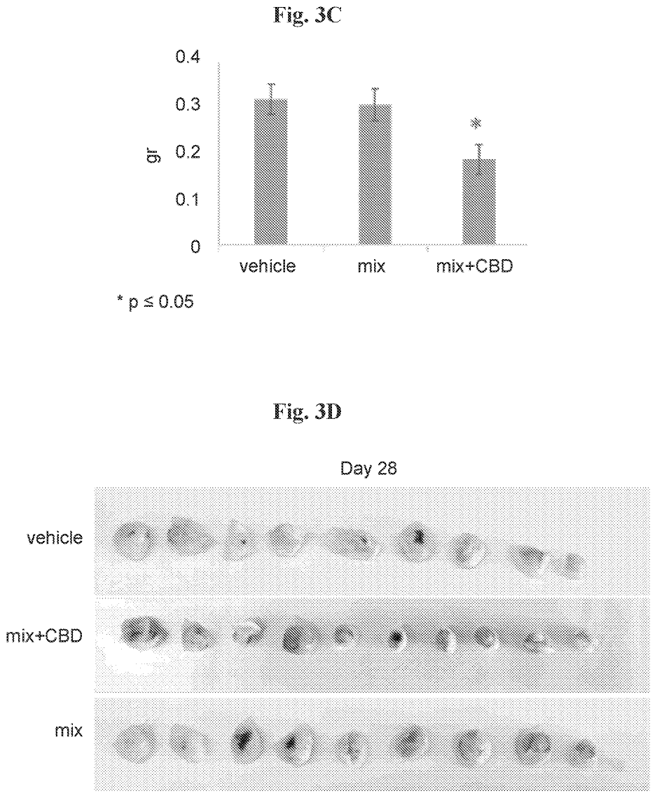

FIG. 3A shows average tumor volume as measured during the experiment (left panel), and tumor volume at Day 27 (right panel), for each one of the three group (Mix, Mix+CBD, and vehicle). FIG. 3B shows the average mouse weight during the experiment, for the three groups vs. a healthy mice, indicating that the treatment per se had no significant effect on the average weight of the mice. FIG. 3C shows the average tumor weight for each one of the three groups at Day 28, indicating a significant difference between the Mix+CBD group and both the Mix group and the vehicle group. FIG. 3D shows pictures of representative tumors extracted from the three groups after termination of the experiment. As clearly demonstrated, the Mix+CBD treatment significantly attenuated tumor progression (reduction of about 42% by weight compared with the control).

### REFERENCES

Jang, J. H. ; Kim, Y. J. ; Lee, H. J. ; Kim, S. W., Journal of Experimental & Clinical Cancer Research, 2012, 31(1), 59
Kim, H. S. ; Kim, M. J. ; Lee, K. H. ; Lee, H. J. ; Kim, S. W., Journal of Ethnopharmacology, 2016, 193, 257-265
Park, J. H. ; Kim, Y. J. ; Lee, H. J. ; Jeong, H. S. ; Kim, S. W., Journal of Agricultural and Food Chemistry, 2011, 59(11), 5933-5939

1. A method for treatment of cancer in a subject in need thereof, comprising administering to said subject a therapeutically effective amount of a combination of sinigrin, vitexin, taraxasterol, and a cannabinoid, wherein said cannabinoid is cannabidiol (CBD) or an enantiomer, diastereomer, or racemate thereof, the ratio between the administered amounts of said sinigrin, said vitexin, and said taraxasterol is in a range of 0.5-2.0: 0.5-2.0: 0.5-2.0, respectively, by weight; and the ratio between the overall administered amount of said sinigrin, said vitexin and said taraxasterol, and the administered amount of said cannabinoid is in a range of 20:1 to 50:1, respectively, by weight.

2. The method of claim 1, wherein the ratio between the administered amounts of said sinigrin, said vitexin, and said taraxasterol is in a range of 0.8-1.2: 0.8-1.2: 0.8-1.2, respectively, by weight; and the ratio between the overall administered amount of said sinigrin, said vitexin and said taraxasterol, and the administered amount of said cannabinoid is in a range of 25:1 to 35:1, respectively, by weight.

3. The method of claim 2, wherein the ratio between the administered amounts of said sinigrin, said vitexin, and said taraxasterol is about 1:1:1, respectively, by weight; and the ratio between the overall administered amount of said sinigrin, said vitexin and said taraxasterol, and the administered amount of said cannabinoid is about 30:1, respectively, by weight.

4. The method of claim 1, wherein said cannabinoid is CBD.

5. The method of claim 1, wherein said sinigrin, said vitexin, said taraxasterol, and said cannabinoid are administered from a sole composition, or from two or more compositions either concomitantly or sequentially at any order.

6. The method of claim 5, wherein each one of said compositions is formulated for oral or parenteral administration.

7. The method claim 1, wherein said cancer is selected from the group consisting of skin cancer, lung cancer, prostate cancer, breast cancer, pancreatic cancer, gastric cancer, colorectal cancer, kidney (renal) cancer, bladder cancer, gallbladder cancer, liver cancer, brain cancer, multiple myeloma, leukemia, lymphoma, and head and neck tumors.

8. The method of claim 7, wherein said skin cancer is melanoma or non-melanoma skin cancer; said breast cancer is selected from the group consisting of luminal A breast cancer, luminal B breast cancer, human epidermal growth factor receptor 2 (HER2)-positive breast cancer, and triple-negative breast cancer; said pancreatic cancer is adenocarcinoma of the pancreas; said colorectal cancer is colorectal carcinoma; said renal cancer is renal carcinoma; said brain cancer is glioblastoma; and said lymphoma is Hodgkin's lymphoma or non-Hodgkin's lymphoma.

9. The method of claim 7, wherein said cancer is breast cancer.

10-18. (canceled)

19. A kit comprising a first composition comprising a cannabinoid; and a second composition comprising sinigrin, vitexin, and taraxasterol, wherein said cannabinoid is CBD, or an enantiomer, diastereomer, or racemate thereof; the ratio between the amounts of said sinigrin, said vitexin, and said taraxasterol is in a range of 0.5-2.0: 0.5-2.0: 0.5-2.0, respectively, by weight;
the ratio between the overall amount of said sinigrin, said vitexin and said taraxasterol, and the amount of said cannabinoid is in a range of 20:1 to 50:1, respectively, by weight; and each one of said compositions is independently formulated for oral or parenteral administration.

20. The kit of claim 19, wherein the ratio between the amounts of said sinigrin, said vitexin, and said taraxasterol is in a range of 0.8-1.2: 0.8-1.2: 0.8-1.2, respectively, by weight;
and the ratio between the overall amount of said sinigrin, said vitexin and said taraxasterol, and the amount of said cannabinoid is in a range of 25:1 to 35:1, respectively, by weight.

21. The kit of claim 20, wherein the ratio between the amounts of said sinigrin, said vitexin, and said taraxasterol is about 1:1:1, respectively, by weight; and the ratio between the overall amount of said sinigrin, said vitexin and said taraxasterol, and the amount of said cannabinoid is about 30:1, respectively, by weight.

22. The kit of claim 19, wherein said cannabinoid is CBD.

23. The kit of claim 19, wherein said first composition is formulated for oral administration, and said second composition is formulated for either oral or parenteral administration.

24. A composition comprising sinigrin, vitexin, taraxasterol, and a cannabinoid, wherein said cannabinoid is CBD, or an enantiomer, diastereomer, or racemate thereof; the ratio between the amounts of said sinigrin, said vitexin, and said taraxasterol in said composition is in a range of 0.5-2.0: 0.5-2.0: 0.5-2.0, respectively, by weight; and the ratio between the overall amount of said sinigrin, said vitexin and said taraxasterol, and the amount of said cannabinoid in said composition is in a range of 20:1 to 50:1, respectively, by weight.

25. The composition of claim 24, wherein the ratio between the amounts of said sinigrin, said vitexin, and said taraxasterol in said composition is in a range of 0.8-1.2: 0.8-1.2: 0.8-1.2, respectively, by weight; and the ratio between the overall amount of said sinigrin, said vitexin and said taraxasterol, and the amount of said cannabinoid in said composition is in a range of 25:1 to 35:1, respectively, by weight.

26. The composition of claim 25, wherein the ratio between the amounts of said sinigrin, said vitexin, and said taraxasterol is about 1:1:1, respectively, by weight; and the ratio between the overall amount of said sinigrin, said vitexin and said taraxasterol, and the amount of said cannabinoid is about 30:1, respectively, by weight.

27. The composition of claim 24, wherein said cannabinoid is CBD.

28. The composition of claim 24, formulated for oral or parenteral administration.

29-32. (canceled)
